(19) United States
(12) Patent Application Publication
(10) Pub. No.: US 2005/0203480 A1
(43) Pub. Date: Sep. 15, 2005

(54) Sanitary undergarment for easily wearing

(76) Inventor: Chin Lung Chang, Taichung (TW)

Correspondence Address: Chin Lung Chang, P.O. Box 63-298, Taichung, 406, TW

(21) Appl. No.: 10/802,123

(22) Filed: Mar. 12, 2004

(51) Int. Cl. A61F 13/15; A61F 13/20

(52) U.S. Cl. 604/402; 604/396

(57) ABSTRACT

A sanitary undergarment includes a panty element having a front panel and a rear panel and a crotch provided between the front panel and the rear panel. A sanitary napkin is disposed on the crotch of the panty element, for engaging onto private parts of users. One or more engaging members are attached onto the front panel and the rear panel of the panty element respectively, and engageable with each other, to attach the panty element onto the users, and to attach the sanitary napkin onto the users without engaging users' legs through the panty element.

### BACKGROUND OF THE INVENTION

1. Field of the Invention

The present invention relates to a sanitary undergarment, and more particularly to a sanitary undergarment having an easily wearing structure for allowing users to easily wear the sanitary undergarments.

2. Description of the Prior Art

Typical sanitary napkins have been widely used in sanitary periods for women, and have to be attached onto the crotch portion of panty elements of undergarments by users themselves. Normally, the sanitary napkins are attached onto the crotch portion of panty elements of undergarments with adhesive layers or materials.

However, the conventional adhesive layers or materials may not solidly attach or secure the sanitary napkins onto the undergarments, such that the sanitary napkins may have a good chance to be moved relative to the undergarments, and thus may not be comfortably worn by the users.

In order to solve the problems, some of typical sanitary undergarments have been developed and comprise a sanitary napkin solidly or firmly attached or secured onto the crotch portion of the panty elements of the undergarments.

However, when wearing the sanitary undergarments, the users have to engage their legs through the leg openings of the sanitary undergarments, and may have a good chance to dirt the sanitary napkins.

In order to further solve the problems, a typical sanitary or hygienic garment assembly has been developed and comprises a backing sheet detachably attached to the panty element as the crotch portion for the panty element, and a sanitary napkin disposed in the backing sheet.

For example, U.S. Pat. No. 4,022,212 to Lovison discloses one of the typical sanitary or hygienic garment assembly which includes a sanitary napkin disposed in a backing sheet which may be detachably attached to the panty element as the crotch portion for the panty element, and for engaging onto the private parts or the pudenda of women.

However, the backing sheet is typically attached to the panty element as the crotch portion with hook-and-loop engaging members which may hurt the users, or may at least make the users feel uncomfortable.

The present invention has arisen to mitigate and/or obviate the afore-described disadvantages of the conventional sanitary undergarments.

### SUMMARY OF THE INVENTION

The primary objective of the present invention is to provide a sanitary undergarment including an easily wearing structure for allowing users to easily wear the sanitary undergarments without engaging their legs through leg openings of the sanitary undergarments.

In accordance with one aspect of the invention, there is provided a sanitary undergarment comprising a panty element including a front panel and a rear panel, and a crotch provided between the front panel and the rear panel thereof, a sanitary napkin disposed on the crotch of the panty element, for engaging onto private parts of users, and a first engaging member and at least one second engaging member attached onto the front panel and the rear panel of the panty element respectively, and engageable with each other, to attach the panty element onto the users, and to attach the sanitary napkin onto the users without engaging users' legs through the panty element.

Each of the front panel and the rear panel includes a free edge having the first and the second engaging members attached thereto respectively. The first engaging member may be extended along a length of the free edge of the front panel. The second engaging member may be attached to ends of the free edge of the rear panel. The first and the second engaging members are preferably the hook-and-loop engaging members.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective view of a sanitary undergarment in accordance with the present invention;

FIG. 2 is a top plan view of the sanitary undergarment;

FIG. 3 is a front plan view illustrating the engagement of the sanitary undergarment onto a user;

FIG. 4 is a rear plan view showing the user and the sanitary undergarment; and

FIG. 5 is a side plan view showing the user and the sanitary undergarment.

### DETAILED DESCRIPTION OF THE PREFERRED EMBODIMENT

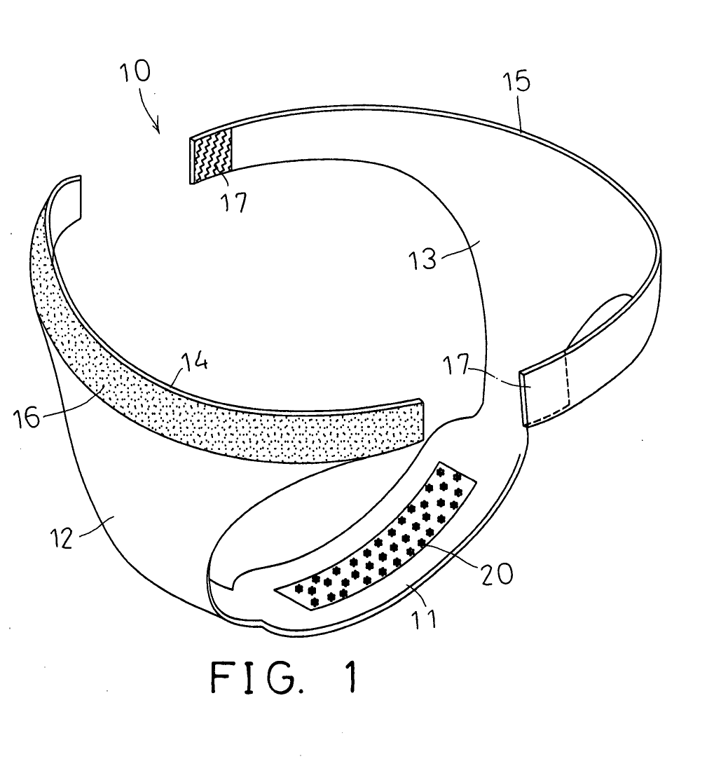

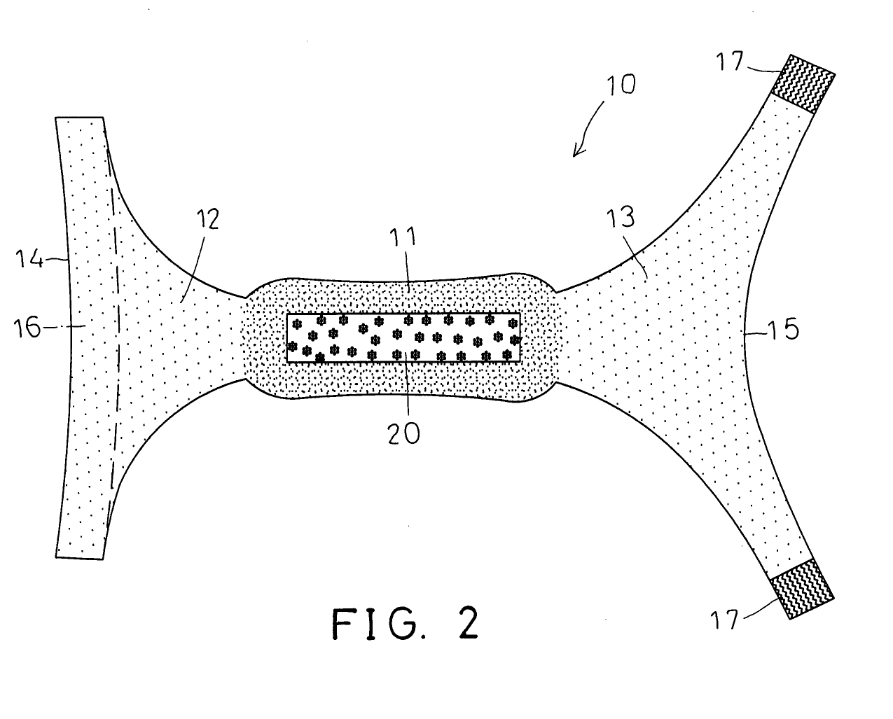

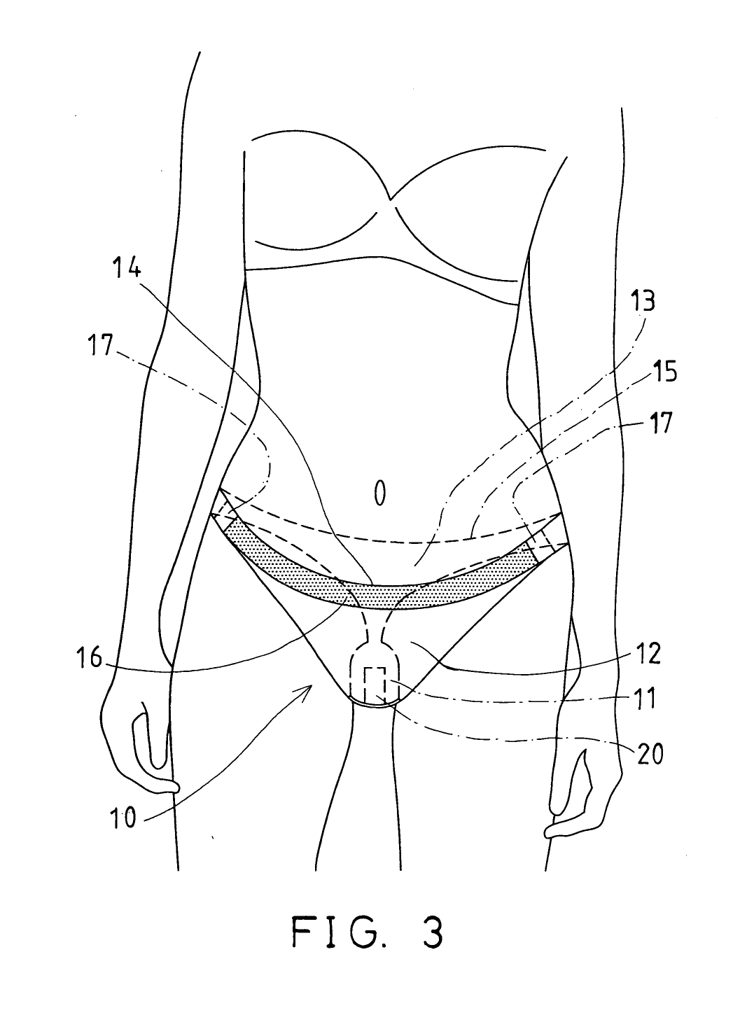

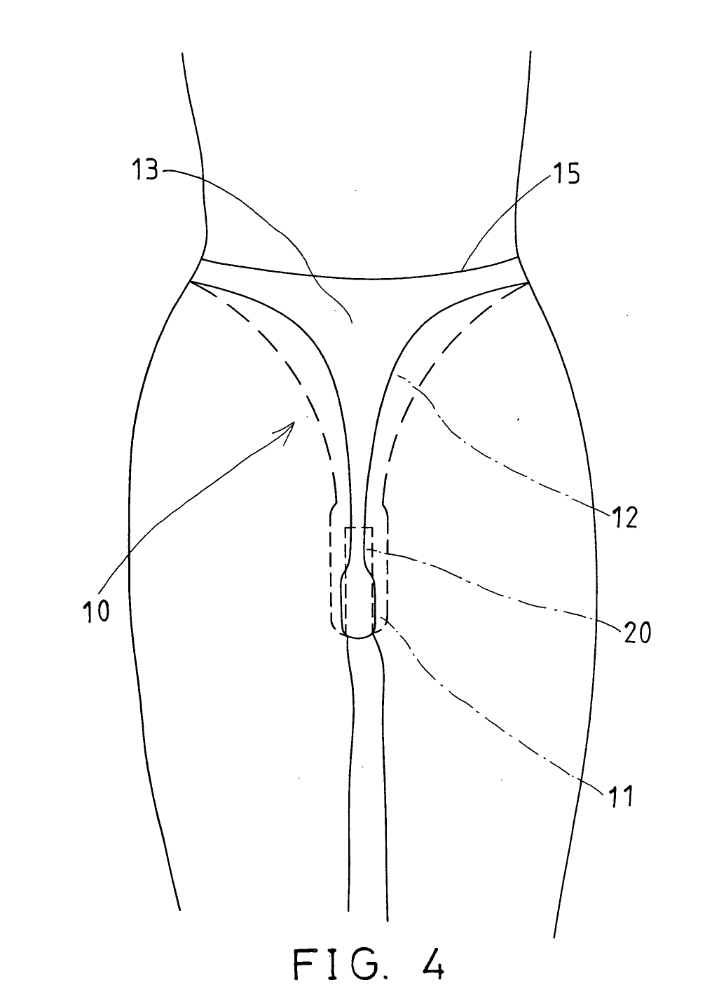

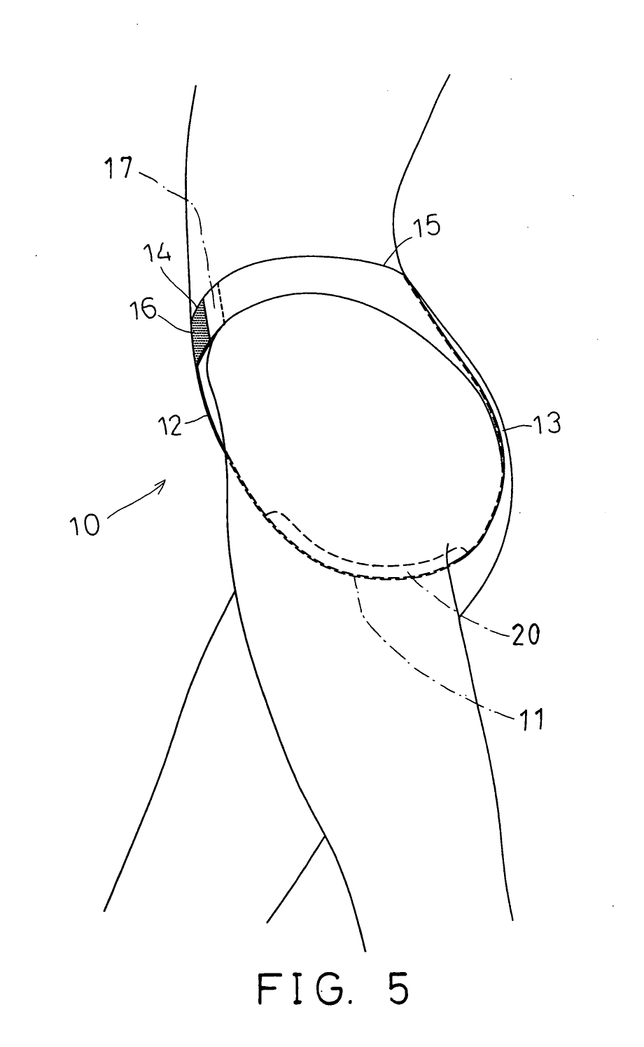

Referring to the drawings, and initially to FIGS. 1-3, a sanitary undergarment in accordance with the present invention comprises a panty element 10 including a crotch 11 formed or provided between a front panel 12 and a rear panel 13, and a sanitary napkin 20 attached or disposed or secured in the crotch 11 of the panty element 10, for engaging onto the private parts or the pudenda of women (FIGS. 3-5).

Each of the front panel 12 and the rear panel 13 of the panty element 10 includes an upper or free portion or edge 14, 15 having one or more engaging members 16, 17, such as hook-and-loop engaging members 16, 17 attached thereto.

For example, the engaging member 16 may be provided or extended along the length of the upper or free portion or edge 14 of the front panel 12 of the panty element 10, and one or more, such as two further engaging members 17 may be provided or disposed on the ends of the upper or free portion or edge 15 of the rear panel 13 of the panty element 10, to engage with the engaging member 16 of the front panel 12, and thus to attach the panty element 10 onto the users (FIGS. 3-5).

It is to be noted that the engaging member 16 of the front panel 12 is faced outwardly or forwardly and will be located in the waist portion of the users, thus will not be engaged between or scratched by the legs of the users, and such that the users will not be hurt or influenced by the engaging members 16, 17.

In addition, the crotch 11 of the panty element 10 and the sanitary napkin 20 may be directly engaged onto the private parts or the pudenda of women, and the panty element 10 may then be attached or secured onto the users with the engaging members 16, 17, without engaging the users' legs through the panty element 10 of the sanitary undergarment, such that the sanitary napkin 20 will not be dirtied by the users.

Accordingly, the sanitary undergarment in accordance with the present invention includes an easily wearing structure for allowing users to easily wear the sanitary undergarments without engaging their legs through leg openings of the sanitary undergarments.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

1. A sanitary undergarment comprising:
a panty element including a front panel and a rear panel, and a crotch provided between said front panel and said rear panel thereof,
a sanitary napkin disposed on said crotch of said panty element, for engaging onto private parts of users, and
a first engaging member and at least one second engaging member attached onto said front panel and said rear panel of said panty element respectively, and engageable with each other, to attach said panty element onto the users, and to attach said sanitary napkin onto the users without engaging users' legs through said panty element.

2. The sanitary undergarment as claimed in claim 1, wherein each of said front panel and said rear panel includes a free edge having said first and said at least one second engaging members attached thereto respectively.

3. The sanitary undergarment as claimed in claim 2, wherein said first engaging member is extended along a length of said free edge of said front panel.

4. The sanitary undergarment as claimed in claim 2, wherein said at least one second engaging member is attached to ends of said free edge of said rear panel.

5. The sanitary undergarment as claimed in claim 2, wherein said first and said at least one second engaging members are hook-and-loop engaging members.
